(19) United States
(12) Patent Application Publication
(10) Pub. No.: US 2014/0254757 A1
(43) Pub. Date: Sep. 11, 2014

(54) X-RAY IMAGING APPARATUS AND CONTROL METHOD FOR THE SAME

(71) Applicant: Samsung Electronics Co., Ltd., Suwon-si (KR)

(72) Inventors: Hyun Hwa OH, Hwaseong-si (KR); Dong Goo KANG, Suwon-si (KR); Sung Hoon KANG, Suwon-si (KR); Young Hun SUNG, Hwaseong-si (KR)

(73) Assignee: SAMSUNG ELECTRONICS CO., LTD., Suwon-si (KR)

(21) Appl. No.: 14/197,716

(22) Filed: Mar. 5, 2014

(30) Foreign Application Priority Data
Mar. 5, 2013 (KR) 10-2013-0023605

(51) Int. Cl. A61B 6/00

(52) U.S. Cl. CPC A61B 6/484
USPC 378/62

(57) ABSTRACT

Disclosed are an X-ray imaging apparatus, which may acquire different phase contrast image signals on a per energy band basis simultaneously without moving a detector or emitting X-rays multiple times by using a photon counting detector that separates detected X-rays into a plurality of energy bands, and a control method for the same. The X-ray imaging apparatus includes an X-ray source which is configured to generate X-rays and emit the X-rays toward a subject, an X-ray detector which is spaced apart from the subject by a predetermined distance and configured to detect X-rays which have propagated through the subject, and to separate the detected X-rays into a plurality of energy bands in order to acquire phase contrast image signals on a per energy band basis, and an image processor which is configured to form a phase contrast image of the subject by using the acquired phase contrast image signals.

### CROSS-REFERENCE TO RELATED APPLICATION

This application claims priority from Korean Patent Application No. 10-2013-0023605, filed on Mar. 5, 2013 in the Korean Intellectual Property Office, the disclosure of which is incorporated herein by reference in its entirety.

### BACKGROUND

1. Field

Exemplary embodiments relate to an X-ray imaging apparatus for forming an X-ray image by causing X-rays to propagate through a subject, and a control method for the same.

2. Description of the Related Art

An X-ray imaging apparatus may be used for forming an image of the internal structure of a subject by emitting X-rays toward the subject and detecting X-rays which have propagated through the subject.

Conventionally, based on the fact that attenuation or absorption of X-rays varies according to constituent substances of a subject, the internal structure of the subject has been imaged by using the intensity of the X-rays which have propagated through the subject.

Considering properties of X-rays, X-rays undergo refraction and interference due to constituent substances of a subject when propagating through the subject, which causes phase shifts thereof. Such phase shifts depend on properties of constituent substances. In recent years, technologies for imaging the interior of a subject by using phase contrast of X-rays have been developed.

X-rays have a greater phase-shift coefficient than an absorption coefficient on a per substance basis. Therefore, phase contrast imaging technologies enable acquisition of a high-contrast image with minimal X-ray exposure, and much research associated with the same is needed.

### SUMMARY

Therefore, it is an aspect of one or more exemplary embodiments to provide an X-ray imaging apparatus which may acquire respective phase-contrast image-signals having different properties on a per energy band basis at a single time by using a photon counting detector that separates detected X-rays on a per energy band basis, without moving the detector or emitting X-rays multiple times, and a control method for the same.

Additional aspects of the exemplary embodiments will be set forth in part in the description which follows and, in part, will be obvious from the description, or may be learned by practice of the exemplary embodiments.

In accordance with one aspect of one or more exemplary embodiments, an X-ray imaging apparatus for forming a phase contrast image includes an X-ray source which is configured to generate X-rays and to emit the generated X-rays toward a subject, an X-ray detector which is spaced apart from the subject by at least a predetermined distance and which is configured to detect X-rays which have propagated through the subject and to separate the detected X-rays into a respective plurality of energy bands in order to acquire respective phase contrast image signals on a per energy band basis, and an image processor which is configured to form a phase contrast image of the subject by using the acquired phase contrast image signals.

The X-ray detector may be further configured to count a number of photons which have an energy which is greater than or equal to a respective threshold energy level which corresponds to a respective one of the plurality of energy bands, from among photons included in the detected X-rays.

The X-ray source may have a focal spot which is used to generate the spatially coherent X-rays. The focal spot may have a diameter having a length of between 2 micrometers and 100 micrometers.

In accordance with another aspect of one or more exemplary embodiments, a control method which is executable by using an X-ray imaging apparatus for forming a phase contrast image includes generating X-rays and emitting the generated X-rays toward a subject, detecting X-rays which have propagated through the subject by using an X-ray detector which is spaced apart from the subject by at least a predetermined distance, acquiring respective phase contrast image signals on a per energy band basis by separating the detected X-rays into a respective plurality of energy bands, and forming a phase contrast image of the subject by using the acquired phase contrast image signals.

The acquiring the respective phase contrast image signals on a per energy band basis may include counting a respective number of photons which have an energy which is greater than or equal to a respective threshold energy level which corresponds to a respective one of the plurality of energy bands, from among photons included in the detected X-rays.

The control method may further include obtaining a pre-shot image of the subject, and controlling at least one condition to be applied to a main shot by analyzing the obtained pre-shot image.

The controlling the at least one condition to be applied to the main shot may include determining at least one property of the subject by analyzing the pre-shot image, and controlling a length of a diameter of a focal spot of an X-ray source based on the determined at least one property of the subject.

The controlling the at least one condition to be applied to the main shot may further include controlling at least one of a distance between the X-ray source and the subject and a distance between the subject and the X-ray detector based on at least one of the determined at least one property of the subject, the length of the diameter of the focal spot of the X-ray source, and a field of view of the X-ray source.

### BRIEF DESCRIPTION OF THE DRAWINGS

These and/or other aspects of the present inventive concept will become apparent and more readily appreciated from the following description of exemplary embodiments, taken in conjunction with the accompanying drawings of which:

FIG. 1 is a view which schematically illustrates phenomena which occur when X-rays propagate through a subject;

FIG. 2 is a view which schematically illustrates an acquisition of an X-ray image by using X-ray attenuation;

FIG. 3 is a graph which illustrates X-ray attenuation and sensitivity to phase shift;

FIG. 4A is a view which diagrammatically illustrates internal constituent substances of the breast, and FIG. 4B is a graph which illustrates attenuation coefficients of internal constituent substances of the breast;

FIG. 5A is a view which schematically illustrates an acquisition of a phase contrast image, and FIG. 5B is a view which schematically illustrates an acquisition of a phase contrast image while an X-ray detector is being moved;

FIG. 6 is a block diagram which illustrates an X-ray imaging apparatus according to an exemplary embodiment;

FIG. 7 is a view which schematically illustrates a configuration of an X-ray detector included in the X-ray imaging apparatus according to an exemplary embodiment;

FIG. 8A is a view which schematically illustrates a configuration of a single pixel of the X-ray detector shown in FIG. 7;

FIG. 8B is a view which schematically illustrates a configuration of a single pixel which may separate detected X-rays into a plurality of energy bands;

FIG. 9A is a graph which illustrates an energy spectrum of X-rays emitted from an X-ray source, and FIG. 9B is a graph which illustrates an energy spectrum separated by the X-ray detector;

FIG. 10 is a view which illustrates an external appearance of an X-ray imaging apparatus according to an exemplary embodiment;

FIG. 11 is a view which geometrically illustrates positions of the subject and the X-ray detector for explanation of phase retrieval.

FIG. 12 is a control block diagram of an X-ray imaging apparatus that may control imaging conditions via auto-exposure control, according to an exemplary embodiment;

FIG. 13 is a view which illustrates a configuration of an X-ray tube included in an X-ray source;

FIG. 14 is a flowchart which illustrates a control method which is executable by using an X-ray imaging apparatus according to an exemplary embodiment; and

FIG. 15 is a flowchart which illustrates a control method which is executable by using an X-ray imaging apparatus that controls imaging conditions via auto-exposure control, according to an exemplary embodiment.

### DETAILED DESCRIPTION

Hereinafter, an X-ray imaging apparatus and a control method for the same according to exemplary embodiments of one aspect will be described with reference to the accompanying drawings.

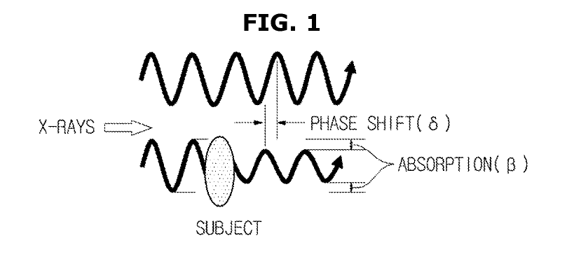

FIG. 1 is a view which schematically illustrates phenomena which occur when X-rays propagate through a subject.

Assuming that X-rays having both particle and wave properties are electromagnetic waves, as exemplarily shown in FIG. 1, X-rays undergo amplitude reduction and phase shift 6 while propagating through a subject. Amplitude reduction of X-rays is caused because of absorption of X-rays by constituent substances of a subject (X-ray absorption β). This amplitude reduction of X-rays is referred to as X-ray attenuation.

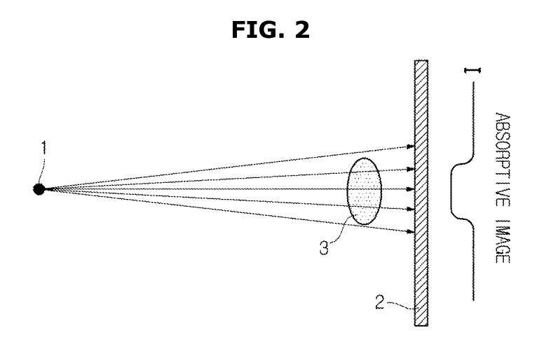

FIG. 2 is a view which schematically illustrates an acquisition of an X-ray image by using X-ray attenuation.

Constituent substances of a subject exhibit different respective X-ray attenuation properties, i.e. different X-ray absorption. Conventionally, the interior of a subject has been imaged by using X-ray attenuation. In the following description of the exemplary embodiments, an image using X-ray attenuation is referred to as an absorptive image. To form the absorptive image, as exemplarily shown in FIG. 2, an X-ray source 1 emits X-rays toward a subject 3, and an X-ray detector 2, which is located nearby with respect to the subject 3, detects X-rays which have propagated through the subject 3. The intensity of the detected X-rays contains information which relates to X-ray attenuation, and thus an absorptive image of the subject 3 may be formed by using the intensity of the detected X-rays.

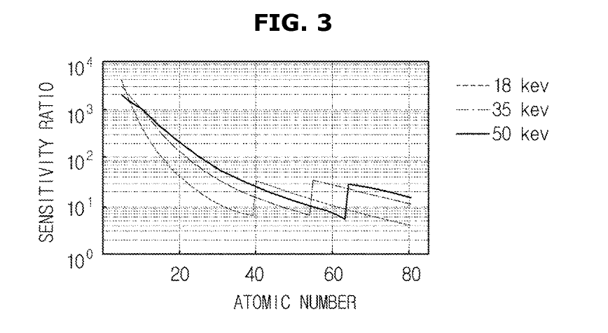

FIG. 3 is a graph which illustrates X-ray attenuation and sensitivity to phase shift.

Phase shift of X-rays occurs because constituent substances of a subject cause refraction and interference of X-rays while X-rays are propagating through the subject. Assuming that the index indicating X-ray attenuation is β and the index indicating phase shift of X-rays is δ, a sensitivity ratio that is a ratio of the two coefficients (δ/β) may be represented as exemplarily shown in FIG. 3. Referring to FIG. 3, it will be appreciated that the phase shift of X-rays is thousands of times more sensitive than X-ray attenuation, although the sensitivity ratio varies according to constituent substances of the subject and energy levels of X-rays.

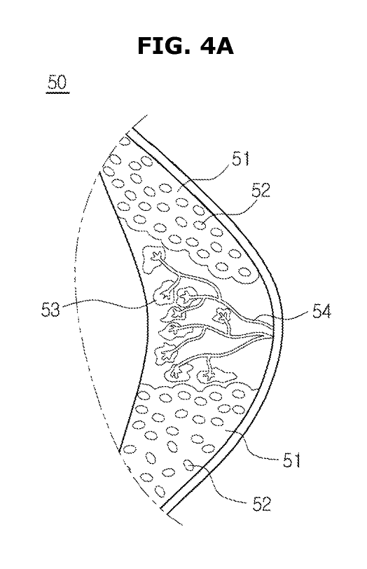

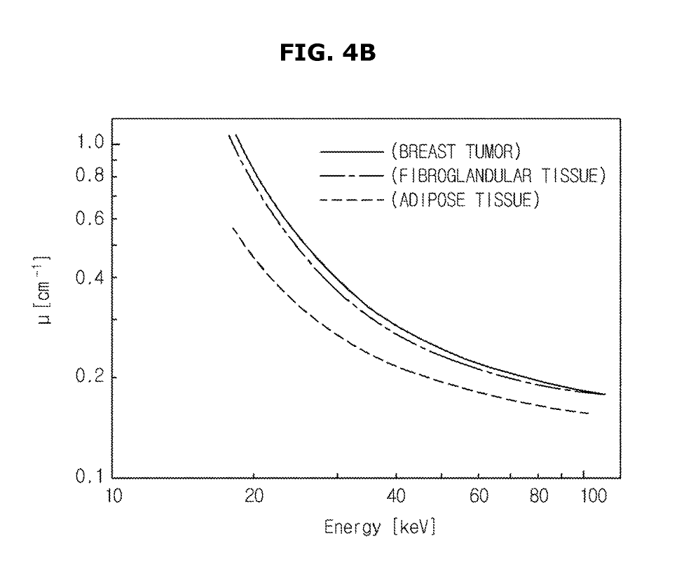

FIG. 4A is a view which diagrammatically illustrates internal constituent substances of the breast, and FIG. 4B is a graph which illustrates attenuation coefficients of internal constituent substances of the breast.

Referring to FIG. 4A, tissues of the breast 50 include fibrous tissues 51 constituting the periphery of the breast for shape maintenance, adipose tissues 52 which are distributed throughout the breast, mammary glands 53 for generation of breast milk, and lactiferous ducts 54 which are used for movement passage of breast milk. Of these tissues, the mammary glands 53 and the lactiferous ducts 54, which relate to generation and supply of breast milk, are referred to as fibroglandular tissues of the breast. As exemplarily shown in FIG. 4B, the fibroglandular tissues exhibit an X-ray attenuation coefficient which is similar to that of lesions, such as tumors, etc.

In addition, because the breast is composed of soft tissue alone, as exemplarily shown in FIG. 4B, internal constituent substances of the breast have a relatively small difference in X-ray attenuation. Thus, it may be difficult to acquire accurate information regarding internal constituent substances of the breast from an absorptive image alone.

As exemplarily shown in FIG. 3, the phase shift of X-rays is typically dozens of times to thousands of times more sensitive than X-ray attenuation. Therefore, with regard to a subject which has a relatively small difference in X-ray attenuation between constituent substances thereof, such as the breast, acquisition of a more vivid and distinguishable X-ray image may be possible by using the phase shift property of X-rays.

Imaging the interior of a subject based on the theory that respective constituent substances of a subject exhibit different phase shifts of X-rays is referred to as phase contrast imaging, and an image which is formed via phase contrast imaging is referred to as a phase contrast image.

Such a phase contrast image may be formed via at least one of interferometry, diffraction-enhanced imaging, in-line phase contrast imaging, and grating interferometry, for example. In particular, in-line phase contrast imaging may be realized by using a configuration similar to that of a general X-ray imaging apparatus without requiring additional optical elements, such as a diffraction lattice or a reflector. An X-ray imaging apparatus according to one exemplary embodiment is designed to acquire a phase contrast image by using in-line phase contrast imaging.

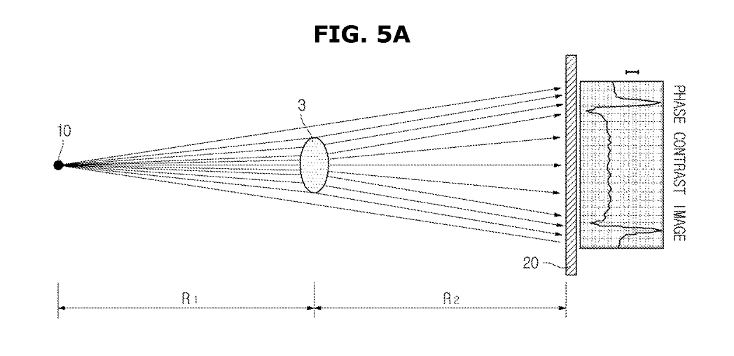

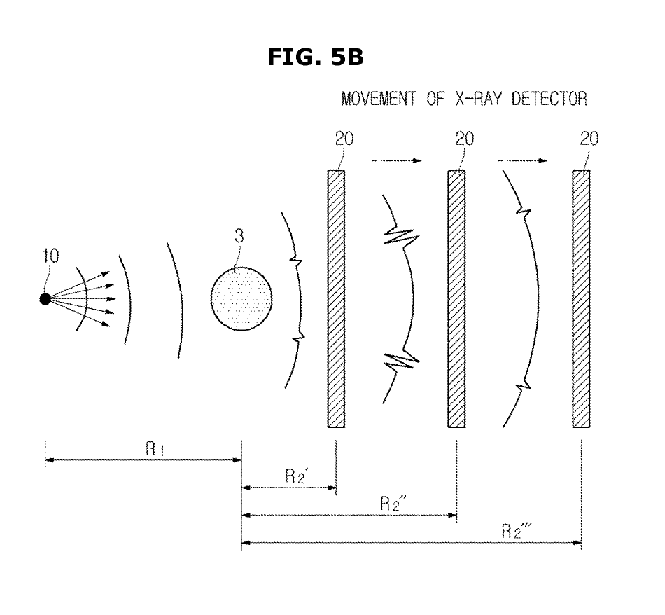

FIG. 5A is a view which schematically illustrates an acquisition of a phase contrast image, and FIG. 5B is a view which schematically illustrates an acquisition of a phase contrast image while an X-ray detector is being moved.

In in-line phase contrast imaging, as exemplarily shown in FIG. 5A, an X-ray detector 20 is spaced from the subject 3 by a distance R2, and the subject 3 is spaced from an X-ray source 10 by a distance R₁. If the X-ray source 10 emits X-rays toward the subject 3, the emitted X-rays first propagate through the subject 3 and thereafter are detected by the X-ray detector 20 that is spaced from the subject 3 by the distance R₂. In an exemplary embodiment, R₁ and R₂ may be determined according to properties of the subject 3 and/or according to X-ray imaging conditions.

A space between the subject 3 and the X-ray detector 20 is called a free space. While X-rays which have propagated through the subject 3 are propagating in the free space, the phase shift of the X-rays is reflected in the intensity of X-rays detected by the X-ray detector 20. In particular, if the subject 3 is spaced from the X-ray detector 20 by at least a given distance such that a free space is present therebetween, information which relates to a phase shift of X-rays that occurs as X-rays propagate through the subject 3 is reflected in the intensity of detectable X-rays.

Note that information which relates to various phase shifts having different properties may be necessary in order to acquire a phase contrast image via in-line phase contrast imaging. As exemplarily shown in FIG. 5B, there are wavefronts having different distortion degrees depending on propagation distances R2′, R2″, R2′″ of X-rays in the free space. This means that the respective phase shift is reflected, by different degrees, in the respective intensities of X-rays. In particular, different properties of phase shifts are reflected in the respective intensity of X-rays according to the corresponding distance between the subject 3 and the X-ray detector 20. Accordingly, as exemplarily shown in FIG. 5B, X-ray detection is implemented while changing a position of the X-ray detector 20 two or more times in order to acquire a plurality of phase contrast image signals having different properties, and a phase contrast image is formed by using the acquired phase contrast image signals.

However, in the case of forming an image while changing a position of the X-ray detector 20, motion artifacts may occur due to movement of the subject 3, and the subject 3 may be excessively exposed to radiation, because X-ray imaging must be implemented multiple times.

Accordingly, the X-ray imaging apparatus according to one or more exemplary embodiments is designed to acquire a phase contrast image of the subject by implementing X-ray imaging at a single position, rather than by moving the X-ray detector.

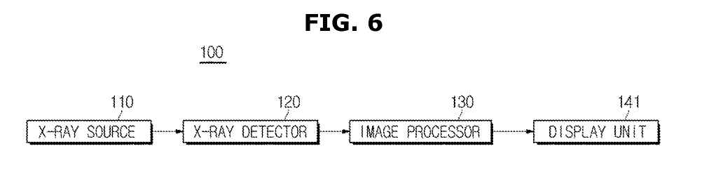

FIG. 6 is a control block diagram which illustrates an X-ray imaging apparatus according to an exemplary embodiment.

Referring to FIG. 6, the X-ray imaging apparatus, designated by reference number 100, includes an X-ray source 110 that generates and emits X-rays toward a subject, an X-ray detector 120 that detects X-rays which have propagated through the subject at a position which is spaced apart from the subject by at least a predetermined distance in order to acquire image signals, an image processor 130 that generates a phase contrast image of the subject by using the acquired image signals, and a display unit 141 that displays the acquired phase contrast image. The X-ray detector 120 is spaced apart from the subject by at least a predetermined distance and contains information which relates to phase contrast of X-rays. Thus, in the following description of exemplary embodiments, a signal which relates to the intensity of X-rays output from the X-ray detector 120 is referred to as a phase contrast image signal, because a phase contrast image may be formed by using signals on a per pixel basis which are output from the X-ray detector 120.

The X-ray source 110 generates X-rays upon receiving electric power from a power supply unit (not shown). The energy level of the X-rays may be controlled by tube voltage, and X-ray intensity or dose may be controlled by tube current and X-ray exposure time.

If X-rays to be emitted have an energy level which falls within a predetermined energy band, the energy band may be defined by an upper limit and a lower limit. The upper limit of the energy band, i.e. the maximum energy level of X-rays to be emitted, may be adjusted based on the magnitude of tube voltage. Also, the lower limit of the energy band, i.e. the minimum energy level of X-rays to be emitted, may be adjusted by using a filter which may be provided inside or outside of the X-ray source 110. Filtering a low energy band of X-rays by using the filter may increase an average energy level of X-rays to be emitted.

The X-ray source 110 may emit monochromatic X-rays or polychromatic X-rays. In the present exemplary embodiment, the X-ray source 110 emits X-rays having an energy level which falls within a predetermined energy band, and the predetermined energy band includes a plurality of energy sub-bands.

To form a phase contrast image, it may be necessary for all X-rays to have the same phase. X-rays having the same phase are referred to as spatially coherent X-rays. Accordingly, the X-ray source 110 may be embodied as a device that generates synchrotron radiation, X-ray laser, or high-order harmonics that have great spatial coherence, or may be embodied as a point source, a focal spot of which is reducible by using a general X-ray tube.

The X-ray detector 120 detects X-rays which have propagated through the subject, and converts the detected X-rays into electric signals in order to acquire phase contrast image signals.

In general, X-ray detectors may be classified based on material composition, conversion from detected X-rays into electric signals, and image signal acquisition.

First, X-ray detectors may be classified into a single device mode and a hybrid device mode according to material composition.

In the case of the single device mode, a part that detects X-rays in order to generate electric signals and a part that reads out and processes electric signals may be formed of a single semiconductor material, or may be fabricated via a single process. For example, a single light receiving device, such as a Charge Coupled Device (CCD) or a Complementary Metal Oxide Semiconductor (CMOS), may be used.

In the case of the hybrid device mode, a part that detects X-rays in order to generate electric signals and a part that reads out and processes electric signals may be formed of different materials, or may be fabricated via different processes. For example, the hybrid device mode may include the case in which X-rays are detected by a light receiving device, such as a photodiode or a cadmium zinc telluride (CdZnTe) detector, and electric signals are read out and processed by a CMOS Read Out Integrated Circuit (ROIC), the case in which X-rays are detected by a strip detector and electric signals are read out and processed by a CMOS ROIC, and the case of using an amorphous silicon (a-Si) or an amorphous selenium (a-Se) flat panel system.

In addition, X-ray detectors may be classified into a direct conversion mode and an indirect conversion mode, according to a respective mode of conversion from X-rays into electric signals.

In the case of the direct conversion mode, electron-hole pairs are temporarily generated in a light receiving device if X-rays are emitted, and as a result of an electric field which is created around both ends of the light receiving device, electrons move to an anode and holes move to a cathode. An X-ray detector converts this movement into electric signals. In the direct conversion mode, the light receiving device may be formed, for example, of any one or more of a-Se, CdZnTe, HgI₂, PbI₂, etc.

In the case of the indirect conversion mode, a scintillator is provided between a light receiving device and an X-ray source, and if photons having a visible light wavelength are discharged via reaction between X-rays emitted from the X-ray source and the scintillator, the light receiving device senses the photons and converts the same into electric signals. In the indirect conversion mode, the light receiving device may be formed, for example, of a-Si, etc., and the scintillator may include any one or more of a thin-film shaped gadolinium oxysulfide (GADOX) scintillator, a micro-column shaped or needle shaped cesium iodide (CSI(T1)), etc.

In addition, X-ray detectors are classified, according to a respective mode of acquisition of image signals, into a charge integration mode in which a signal is acquired from charges after the charges are stored for a predetermined time, and a photon counting mode in which photons having an energy level which is greater than or equal to a threshold energy level are counted whenever a signal is generated by a single X-ray photon.

The X-ray imaging apparatus 100 according to an exemplary embodiment may be configured to separate X-rays emitted from the X-ray detector 120 into a plurality of energy bands, even when X-rays are emitted only once, based on the photon counting mode that causes less X-ray exposure with respect to the subject and less noise which is associated with an X-ray image than the charge integration mode.

Although there is no limit as to material composition and electric signal conversion of the X-ray detector 120, for convenience of description, an exemplary embodiment which employs the direct conversion mode in which electric signals are directly acquired from X-rays and a hybrid mode in which a light receiving device for detection of X-rays and a readout circuit chip are coupled to each other will be described in detail.

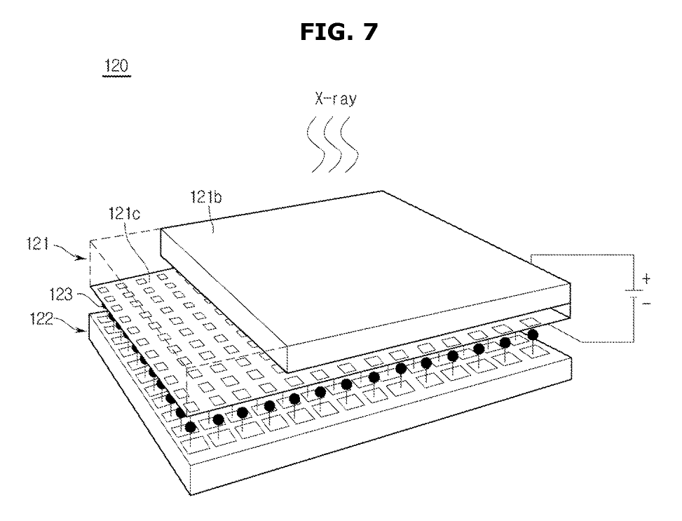

FIG. 7 is a view which schematically illustrates a configuration of the X-ray detector included in the X-ray imaging apparatus, according to an exemplary embodiment.

Referring to FIG. 7, the X-ray detector 120 includes a light receiving device 121 which is configured to convert detected X-rays into electric signals, and a readout circuit 122 which is configured to read out the electric signals. In particular, the readout circuit 122 takes the form of a 2D pixel array which includes a plurality of pixels. The light receiving device 121 may be formed of a monocrystalline semiconductor material in order to achieve high dynamic level, high resolution and fast response at a low energy level and a low dose. Examples of the monocrystalline semiconductor material may include any one or more of Ge, CdTe, CdZnTe, and GaAs.

The light receiving device 121 may take the form of a PIN photodiode in which a p-type layer 121c in the form of a 2D pixel array is bonded to the bottom of a high-resistance n-type semiconductor substrate 121b. The readout circuit 122 is formed of a CMOS and is coupled to the light receiving device 121 on a per pixel basis. The CMOS readout circuit 122 and the light receiving device 121 may be bonded to each other via flip-chip bonding, as bumps 123, which may be formed, for example, of PbSn, In, etc. are reflow soldered and thermally pressed. Of course, the above-described configuration is provided as one example of the X-ray detector 120, and the configuration of the X-ray detector 120 is not limited to the above description.

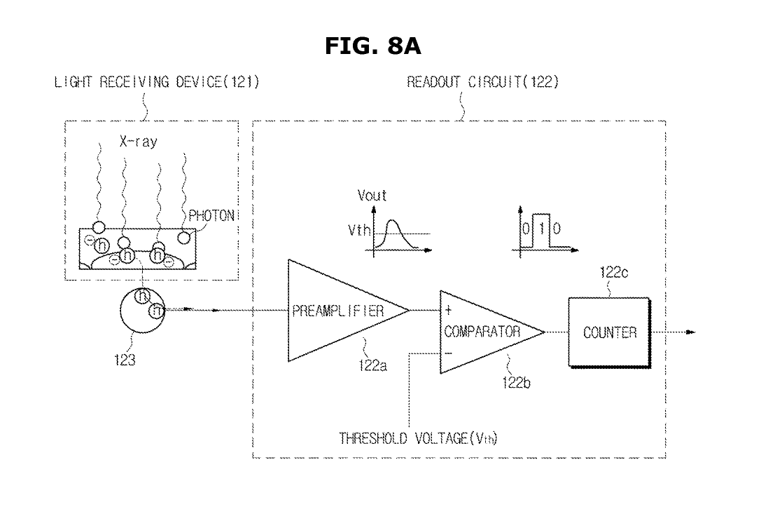

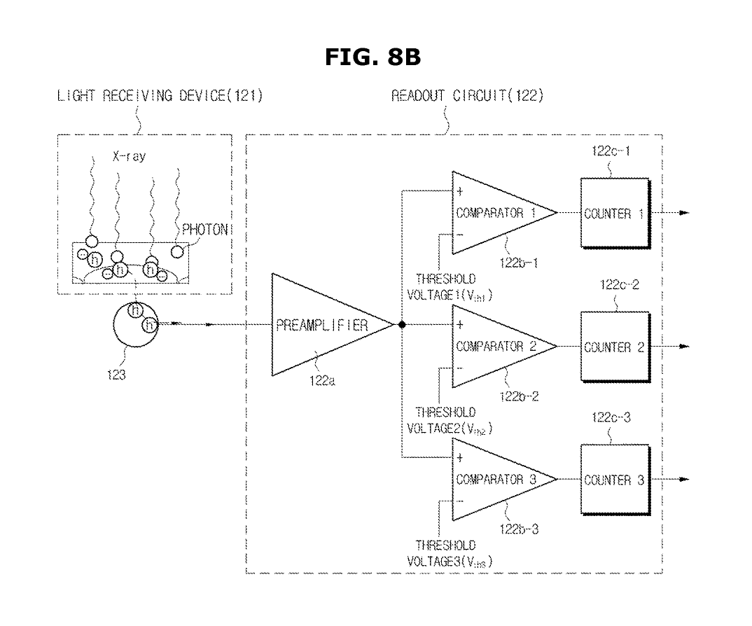

FIG. 8A is a view which schematically illustrates a configuration of a single pixel of the X-ray detector shown in FIG. 7, and FIG. 8B is a view which schematically illustrates a configuration of a single pixel which may separate detected X-rays into a plurality of energy bands.

Referring to FIG. 8A, if photons of X-rays are introduced into the light receiving device 121, electrons of a valence band receive the energy of the photons and are excited to a conduction band beyond a band gap energy difference. This results in generation of electron-hole pairs in a depletion region.

If metal electrodes are provided respectively at a p-type layer and an n-type substrate of the light receiving device 121 and a reverse bias is applied to the metal electrodes, electrons from among the electron-hole pairs generated in the depletion region are dragged to an n-type region, and holes are dragged to a p-type region. Then, as the holes dragged to the p-type region are input to the readout circuit 122 through the bonding bumps 123, a readout of electric signals generated by the photons may be possible. However, the electrons may be input to the readout circuit 122 so as to generate electric signals according to a configuration of the light receiving device 121 and the applied voltage, for example.

The readout circuit 122 may take the form of a 2D pixel array of the light receiving device 121 which corresponds to p-type semiconductors. Thus, the readout circuit 122 reads out electric signals on a per pixel basis. If charges of the light receiving device 121 are input to the readout circuit 122 through the bonding bumps 123, a preamplifier 122a of the readout circuit 122 accumulates the input charges generated per photon, and outputs a corresponding voltage signal.

The voltage signal output from the preamplifier 122a is transmitted to a comparator 122b. The comparator 122b compares the input voltage signal with a threshold voltage that may be controlled from the outside, and outputs a pulse signal of ‘0’ or ‘1’ based on the comparison result. A counter 122c outputs a digital image signal by counting how many times the pulse signal of ‘1’ appears. An X-ray image of the subject may be acquired via combination of image signals on a per pixel basis.

In particular, the threshold voltage corresponds to threshold energy E. To count the number of photons which have an energy level which is greater than or equal to the threshold energy E, a threshold voltage which corresponds to the threshold energy E is input to the comparator 122b. The correspondence between threshold energy and threshold voltage is based on the fact that the magnitude of an electric signal (voltage) generated from the light receiving device is variable according to energy of photons. Thus, a threshold voltage which corresponds to the desired threshold energy may be calculated by using a relational expression between voltage and energy of photons. In the following description of exemplary embodiments, inputting threshold energy to the X-ray detector 120 may refer to inputting a threshold voltage which corresponds to the threshold energy.

In the X-ray imaging apparatus 100 according to an exemplary embodiment, in order to acquire phase contrast image signals having different properties on a per energy band basis, the X-ray source 110 may emit X-rays having a plurality of energy bands, i.e. wideband X-rays once, and the X-ray detector 120 may detect the X-rays in order to separate the same into a plurality of energy bands.

To this end, as exemplarily shown in FIG. 8B, a plurality of comparators and a plurality of counters may be provided in order to count photons which are separated into a plurality of energy bands. Although the exemplary configuration of FIG. 8B includes three comparators, the exemplary embodiment is not limited thereto, and the number of comparators may be determined according to the number of energy bands to be separated.

Referring to FIG. 8B, if electrons or holes generated per photon are input to the preamplifier 122a such that a voltage signal is output, the voltage signal is input to each of three comparators 122b-1, 122b-2, and 122b-3. Then, if each of threshold voltage 1 Vₜₕ₁, threshold voltage 2 Vₜₕ₂, and threshold voltage 3 Vₜₕ₃ is input to the respective comparators, the comparator 1 122b-1 compares the threshold voltage 1 with the input voltage, and a counter 1 counts the number of photons that generate a respective voltage which is greater than the threshold voltage 1. In the same manner, a counter 2 counts the number of photons that generate a respective voltage which is greater than the threshold voltage 2, and a counter 3 counts the number of photons that generate a respective voltage which is greater than the threshold voltage 3.

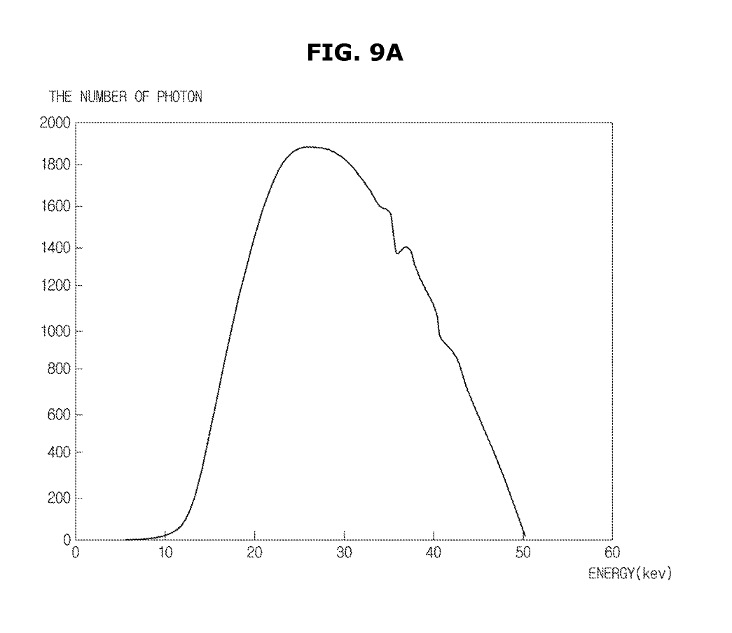

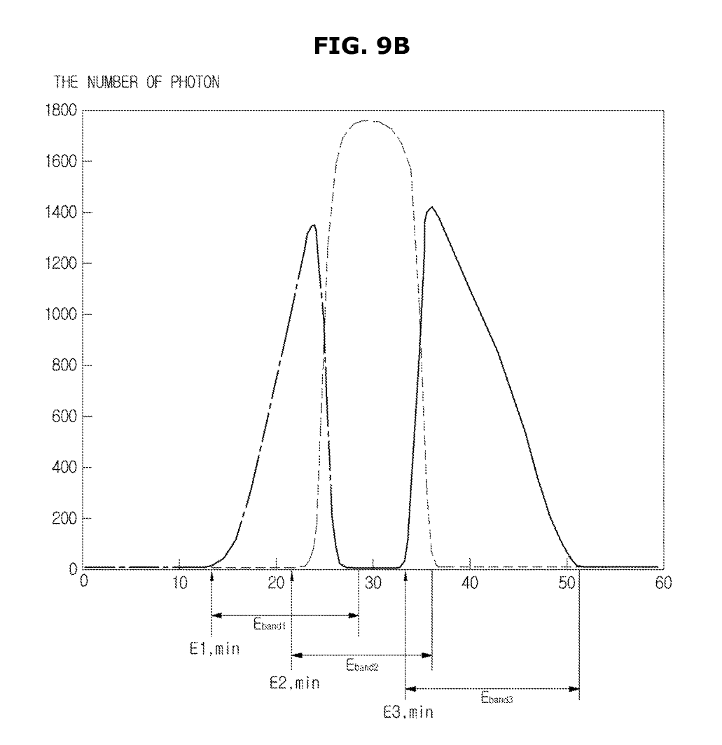

FIG. 9A is a graph which illustrates an energy spectrum of X-rays emitted from the X-ray source, and FIG. 9B is a graph which illustrates an energy spectrum separated by the X-ray detector.

Energy of X-rays emitted from the X-ray source 110 differs on a per subject basis. For example, if the subject is a breast, as exemplarily shown in FIG. 9A, X-rays, for which a lower limit of the energy band is 10 keV and an upper limit of the energy band is 50 keV, may be generated and emitted. To this end, a tube voltage of 50 kVp may be supplied for generation of X-rays, and filtering of a low energy band (approximately 0˜10 kev) of X-rays may be implemented for emission of X-rays. In this case, an X-ray dose (i.e., the number of X-ray photons), as represented by the vertical axis of the graph, may be controlled based on tube current and X-ray exposure time.

The X-ray detector 120 may detect X-rays which are emitted from the X-ray source 110 and separate the detected X-rays into a plurality of energy bands as exemplarily shown in FIG. 9B. In FIG. 9B, X-rays are separated into three energy bands E_{band1}, E_{band2}, and E_{band3}. To this end, the comparator 1 122b-1 of FIG. 8B calculates a voltage which corresponds to E_{1,min} in order to input the same as a threshold voltage, the comparator 2 122b-2 calculates a voltage which corresponds to E_{2,min} in order to input the same as a threshold voltage, and the comparator 3 122b-3 calculates a voltage which corresponds to E_{3,min} in order to input the same as a threshold voltage.

The energy spectrum exemplarily shown in FIGS. 9A and 9B is provided only as one example that may be applied to the X-ray imaging apparatus 100, and the energy bands of X-rays to be emitted and separated by the X-ray imaging apparatus 100 are not limited to the exemplary illustration. As mentioned above, the energy bands of X-rays to be generated and emitted by the X-ray source 110 may vary according to properties of the subject, and the range and number of the energy bands separated by the X-ray detector 120 may vary according to properties of the subject or the definition or resolution of a desired phase contrast image. The greater the number of energy bands to be separated, the greater the edge enhancement, and the greater the definition of the phase contrast image.

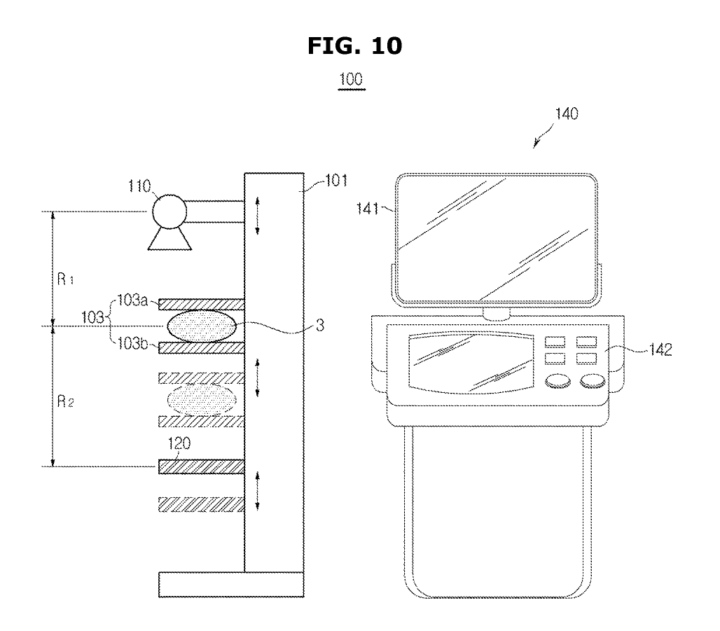

FIG. 10 is a view which illustrates an external appearance of an X-ray imaging apparatus according to an exemplary embodiment. Hereinafter, operations of the X-ray imaging apparatus 100 will be described in detail with reference to FIGS. 10 and 6.

Referring to FIG. 10, the X-ray source 110 and the X-ray detector 120 may be vertically movably mounted to a housing 101. The subject 3 may be fixed with respect to the apparatus 100 by a fixing assembly 103. Likewise, the fixing assembly 103 may be vertically movably mounted to the housing 101, and may include a support plate 103b which is configured to support the subject 3 and a compression paddle 103a which is configured to compress the subject 3.

Although imaging of a subject composed of soft tissues, such as the breast, may need to compress and fix the subject via the fixing assembly 103, some subjects may not need compression or fixing thereof during X-ray imaging. Accordingly, the X-ray imaging apparatus 100 may not include the fixing assembly 103, or may include only the support plate 103b of the fixing assembly 103, according to a subject.

A distance R₁ between the X-ray source 110 and the subject 3 may be controlled by adjusting respective positions of the X-ray source 110 and the fixing assembly 103, and a distance R₂ between the subject 3 and the X-ray detector 120 may be controlled by adjusting respective positions of the fixing assembly 103 and the X-ray detector 120.

Once the distance R₁ between the X-ray source 110 and the subject 3 and the distance R₂ between the subject 3 and the X-ray detector 120 are appropriately set, the X-ray source 110, the fixing assembly 103, and the X-ray detector 120 are fixed at respective positions which correspond to the set distances R₁ and R₂, and then X-ray imaging is implemented.

The X-ray detector 120 may include a Photon Counting Detector (PCD) in order to separate detected X-rays into a plurality of energy bands. As such, phase contrast image signals having different properties may be acquired via single X-ray imaging, without implementing X-ray imaging multiple times while moving the X-ray detector 120. In particular, a difference between the phase contrast image signals is not caused by a distance between the subject 3 and the X-ray detector 120, but is instead caused by an energy band within which each respective one of the phase contrast image signals which are separated on a per energy band basis falls.

If the X-ray detector 120 acquires and outputs a plurality of phase contrast image signals which are separated on a per energy band basis, the image processor 130 forms a phase contrast image of the subject by using the acquired phase contrast image signals. Although the image processor 130 may be provided in a host device 140 that controls general operations of the X-ray imaging apparatus 100, a position of the image processor 130 is not limited to the above description.

The host device 140 includes the display unit 141 that is configured for displaying the image formed by the image processor 130, and an input unit 142 that is configured for receiving a user instruction which relates to an operation of the X-ray imaging apparatus 100.

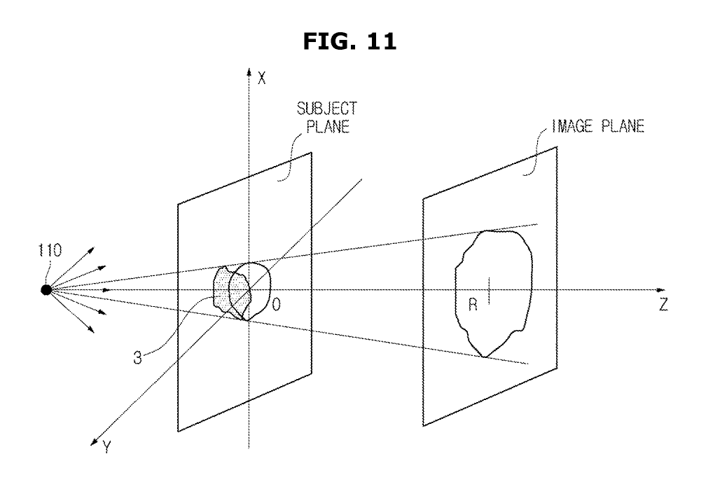

FIG. 11 is a view which geometrically illustrates respective positions of the subject and the X-ray detector for explanation of phase retrieval. Hereinafter, formation of the phase contrast image by the image processor 130 will be described in detail with reference to FIG. 11.

First, the image processor 130 implements phase retrieval from phase contrast image signals output from the X-ray detector 120. To this end, the geometrical relationship as exemplarily shown in FIG. 11 is used. Referring to FIG. 11, it is assumed that the subject and the X-ray detector are located in a 3D space that is defined by x-axis, y-axis and z-axis coordinates, the subject is present on a subject plane, and the X-ray detector 120 is present on an image plane. In particular, it is assumed that the z-axis corresponds to an optical axis along which X-rays propagate, the subject plane is defined such that the z-axis passes the zero point (z=0), and the image plane is defined such that the z-axis passes a point R (z=R).

The intensity I and phase distribution Φ of the detected X-rays may be represented in terms of line integrals of the complex index of refraction. The complex index of refraction n may be defined by or expressible as the following Equation 1.

n(r)=1−δ−iβ Equation 1

In Equation 1, the imaginary number β denotes X-ray absorption or attenuation, and the real number δ denotes a phase shift due to constituent substances of the subject. n satisfies |n−1|<<1, and r is defined as (r_{⊥}, z).

The intensity I and phase distribution Φ of X-rays are defined by or expressible as the following Equation 2 and Equation 3.

I(r_{⊥},0,λ)=exp[−M(r_{⊥},0,λ)] Equation 2
where,

M(r_{⊥},0,λ)=(4π/λ)∫_{-∞}⁰β(r_{⊥},z′,λ)dz′

φ(r_{⊥},0,λ)=−(2π/λ)∫_{-∞}⁰δ(r_{⊥},z′,λ)dz′ Equation 3

where, M denotes absorption or attenuation. Wavelength (λ) dependence of the imaginary number β and the real number δ of the complex index of refraction n may be represented by or expressible as the following Equation 4 and Equation 5.

β(λ)=(λ/λ₀)⁴β(λ₀) Equation 4

δ(λ)=(λ/λ₀)²δ(λ₀) Equation 5

X-ray propagation from the subject plane (z=0) to the image plane (z=R) may be represented by a Fresnel integral. The Fresnel integral may be approximated by the following Equation 6 using a Transport of Intensity Equation (TIE).

(Rλ/2π)[−∇²φ(r_{⊥},0,λ)−∇φ(r_{⊥},0,λ)·∇ ln I(r_{⊥},0,λ)]=I(r_{⊥},0,λ)/I(r_{⊥},0,λ)−1 Equation 6

In Equation 6, if X-ray intensity distribution in the subject plane does not greatly differ from X-ray intensity distribution in the image plane, the right side may be replaced with ln [I(r_{⊥}, R̅, λ)]=ln [I(r_{⊥}, 0, λ)].

Equation 6 may be represented by the following Equation 7 by synthesizing Equations 2, 3, 4, and 5.

−σ³M(r_{⊥},0,λ₀)+γσ(−∇²φ)(r_{⊥},0,λ₀)+γσ⁴∇φ(r_{⊥},0,λ₀)·∇M(r_{⊥},0,λ₀)=ln [I(r_{⊥},0,λ)] Equation 7

where, σ=λ/λ₀ and γ=Rλ/2π. In one example, if the X-ray detector 120 separates phase contrast image signals into three energy bands, i.e., if the phase contrast image signals correspond to three different wavelengths λ₀, λ₁, and λ₂, the following Equation 8 may be expressed as follows.

$$A ( \begin{matrix}M ( {r}_{⊥} , 0 , {λ}_{0} ) \\ - {∇}^{2} ϕ ( {r}_{⊥} , 0 , {λ}_{0} ) \\ ∇ M \cdot ∇ ϕ ( {r}_{⊥} , 0 , {λ}_{0} )\end{matrix} ) = ( \begin{matrix}{F}_{0} \\ {F}_{1} \\ {F}_{2}\end{matrix} ) \\ where , A = ( \begin{matrix}- 1 & {γ}_{0} & {γ}_{0} \\ - {σ}_{1}^{3} & {σ}_{1} {γ}_{1} & {σ}_{1}^{4} {γ}_{1} \\ - {σ}_{2}^{3} & {σ}_{2} {γ}_{2} & {σ}_{2}^{4} {γ}_{2}\end{matrix} ) . \qquad Equation 8$$

In particular, the function of the right side Fᵢ=ln [I(r_{⊥}, R, λᵢ)] may be calculated by using phase contrast image signals with respect to three energy bands output from the X-ray detector 120, i.e., the intensity of X-rays with respect to three energy bands. Thus, M, which represents X-ray attenuation and Laplacian phase distribution, may be acquired as the value of Equation 8, and phase distribution may be retrieved by calculating the Poisson equation as expressed by the following Equation 9.

−∇²φ(r_{⊥},0,λ₀)=ΣA₁ⱼ⁻¹Fⱼ Equation 9

If phase distribution Φ is retrieved, the complex index of refraction n may be determined by applying Equation 1, Equation 2, and Equation 3. The image processor 130 may determine a value of the complex index of refraction n via the above-described procedure, and form a phase contrast image of the subject by using the determined value. The formed phase contrast image of the subject may clearly show the profile of constituent substances of the subject, and may vividly show even small details.

Further, the image processor 130 may implement image calibration for achieving one or more enhancements in the quality of an X-ray image, such as, for example, any one or more of flat field correction, noise reduction, etc. The calibrated phase contrast image of the subject may be displayed via the display unit 141.

The image processor 130 may form an absorptive image which does not contain X-ray phase contrast data. As necessary, the image processor 130 may selectively form an absorptive image or a phase contrast image, or may form both the absorptive image and the phase contrast image in order to display the same via the display unit 141. To form the absorptive image, X-ray imaging may be performed in a state in which the distance between the subject and the X-ray detector 120 becomes zero. In addition, the absorptive image may be formed by using phase contrast image signals which are acquired in a state in which the subject is spaced apart from the X-ray detector 120 by a predetermined distance in order to generate a phase contrast image.

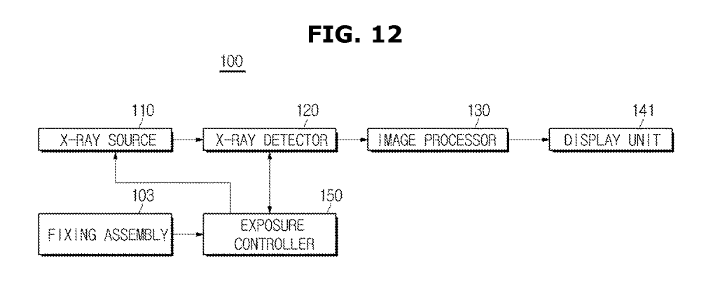

FIG. 12 is a control block diagram of an X-ray imaging apparatus that may control imaging conditions via auto-exposure control, according to an exemplary embodiment.

Referring to FIG. 12, the X-ray imaging apparatus 100 further includes an exposure controller 150 that is configured to set and control one or more imaging conditions by using image signals acquired via a pre-shot. A description of the other components is equal to the above description of FIG. 6 and thus will be omitted hereinafter.

A pre-shot may be implemented in order to enable X-ray imaging conditions which are suitable for properties of the subject to be set. The pre-shot may be implemented in a state in which tube current and X-ray exposure time are adjusted in order to reduce an X-ray dose.

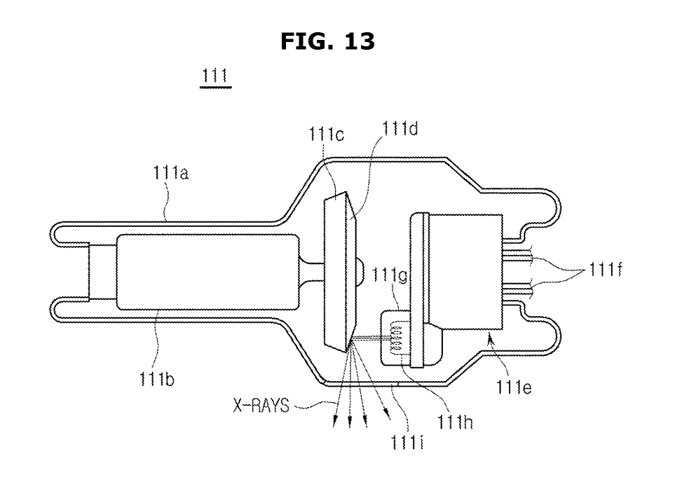

The conditions which are controllable by using the exposure controller 150 may include X-ray generation and emission conditions, and conditions which relate to a distance between the X-ray source 110, the subject, and the X-ray detector 120. First, conditions which relate to X-ray generation and emission will be described with reference to FIG. 13.

FIG. 13 is a view which illustrates a configuration of an X-ray tube included in the X-ray source.

Referring to FIG. 13, the X-ray source 110 includes the X-ray tube 111. The X-ray tube 111 may be embodied as a diode vacuum tube which includes an anode 111c and a cathode 111e. The cathode 111e includes a filament 111h and a focusing electrode 111g which is configured for focusing of electrons. The focusing electrode 111g may also be referred to as a focusing cup.

The interior of a glass tube 111a is evacuated to a pressure of about 10 mmHg, and the filament 111h of the cathode 111e is heated to a high temperature in order to generate thermal electrons. In one example, the filament 111h may be a tungsten filament, and may be heated as current is applied to an electrically conductive wire 111f which is connected to the filament 111h.

The anode 111c may be formed of copper. A target material 111d may be applied to or disposed at one side of the anode 111c facing the cathode 111e. The target material 111d may include a high resistance material, such as any one or more of Cr, Fe, Co, Ni, W, Mo, etc. As the melting point of the target material 111d increases, the size (i.e., the length of the diameter) of the focal spot decreases. In particular, the focal spot refers to an effective focal spot. In addition, the target material 111d is tapered by a predetermined angle. As the tapering angle decreases, the size of the focal spot decreases.

If a high voltage is applied between the cathode 111e and the anode 111c, thermal electrons are accelerated and collide with the target material 111g of the anode 111c, whereby X-rays are generated. The generated X-rays are emitted outward through a window 111i. The window 111i may be formed, for example, of a thin beryllium (Be) film. In this case, a filter may be located at the front side or the rear side of the window 111i in order to filter X-rays which fall within a specific energy band.

The target material 111d may be rotated by a rotor 111b. If the target material 111d is rotated, a heat accumulation rate may be increased by a factor of ten or more on a per unit area basis, and the size of the focal spot may be reduced as compared to the case in which the target material 111d is stationary.

Voltage which is applied between the anode 111c and the cathode 111e of the X-ray tube 111 is referred to as tube voltage, and the magnitude of the tube voltage may be represented as a peak value (kVp). If the tube voltage increases, the velocity of thermal electrons increases, and consequently the energy level of X-rays (the energy level of photons) which are generated via collisions between the thermal electrons and the target material increases. Current which is applied to the X-ray tube 111 is referred to as tube current, and the magnitude of the tube current may be represented as an average value (mA). If the tube current increases, the number of thermal electrons discharged from the filament increases, and consequently X-ray dose (i.e., the number of X-ray photons) which is generated via collisions between the thermal electrons and the target material 111d increases.

Accordingly, the energy level of X-rays may be controlled based on tube voltage, and X-ray intensity or dose may be controlled based on tube current and X-ray exposure time. More specifically, if X-rays to be emitted fall within a predetermined energy band, the energy band may be defined by an upper limit and a lower limit. The upper limit of the energy band, i.e., the maximum energy level of the X-rays to be emitted, may be adjusted based on the magnitude of tube voltage, and the lower limit of the energy band, i.e., the minimum energy level of the X-rays to be emitted, may be adjusted by the filter. Filtering a low energy band of X-rays by using the filter may increase an average energy level of the X-rays to be emitted.

In order to acquire phase contrast image signals with respect to a plurality of energy bands via the X-ray detector 120, the X-ray source 110 may emit polychromatic X-rays, and an energy band of the polychromatic X-rays may be defined by an upper limit and a lower limit.

In one exemplary embodiment, the X-ray imaging apparatus 100 may emit spatially coherent X-rays by using the general X-ray tube 111. For example, if the size of the focal spot is reduced such that the length of the diameter of the focal spot falls within a range of several micrometers to dozens of micrometers (e.g., within a range of between 2 micrometers and 100 micrometers), spatially coherent X-rays may be generated. Although the size of the focal spot is reduced as the melting point and rotation rate of the target material 111d increase and the tapering angle of the target material 111d decreases as described above, the size of the focal spot may vary according to any one or more of tube voltage, tube current, the size of the filament, the size of the focusing electrode, the distance between the anode and the cathode, etc. Accordingly, reducing the size of the focal spot to a range of several micrometers to dozens of micrometers by adjusting controllable ones of the aforementioned conditions may result in generation of spatially coherent X-rays. In addition, the size of the focal spot may vary according to any one or more of various properties of a subject.

The exposure controller 150 may set at least one of several X-ray generation conditions, such as, for example, any one or more of tube voltage, tube current, exposure time, the kind of the target material of the anode, a distance between the anode and the cathode, the kind of the filter, etc. In this case, a pre-shot image of the subject may be obtained and then analyzed in order to set one or more conditions which are optimized to the subject. To this end, the exposure controller 150, as exemplarily shown in FIG. 12, may receive image signals from the X-ray detector 120 and/or from the image processor 130. In addition, as exemplarily shown in FIG. 10, if the subject 3 is compressed by the fixing assembly 103, the exposure controller 150 may receive information which relates to a distance between the compression paddle 103a and the support plate 103b, thereby setting one or more conditions which relate to the thickness of the subject 3.

The exposure controller 150 may set and control a distance R₁ between the X-ray source 110 and the subject 3 and a distance R₂ between the subject 3 and the X-ray detector 120. To this end, the exposure controller 150 may understand one or more properties of constituent substances of the subject by analyzing a pre-shot image, and may set the appropriate distances R₁ and R₂ based on the size of the focal spot of the X-ray source 110, one or more properties of the subject including the thickness of the subject, and the Field of View (FOV) of X-rays. As exemplarily shown in FIG. 10, the X-ray source 110, the fixing assembly 103, and the X-ray detector 120 are vertically movable on the housing 101, and therefore may be moved in response to one or more control signals with respect to the set distances R₁ and R₂. In this way, the distances may be adjusted to values which are optimized to the size of the focal spot of the X-ray source 110, any one or more among properties of the subject, the Field of View (FOV) of X-rays, etc.

In the above-described exemplary embodiment, the X-ray source 110 emits X-rays once and the X-ray detector 120 detects and separates the emitted X-rays in order to acquire different phase contrast image signals on a per energy band basis. As such, it is unnecessary to emit X-rays multiple times, which may reduce X-ray exposure and may obviate a need for movement of the X-ray detector 120, thereby preventing deterioration in the quality of an image due to motion artifacts.

According to another exemplary embodiment, the X-ray source 110 emits X-rays which respectively fall within different energy bands and the X-ray detector 120 detects the respective X-rays in order to acquire respective phase contrast image signals having correspondingly different properties. The present exemplary embodiment may prevent deterioration in the quality of an image due to motion artifacts because the X-ray detector 120 is not moved, although X-rays are emitted multiple times.

Hereinafter, exemplary embodiments with regard to a control method for the X-ray imaging apparatus according to one or more aspects of the present inventive concept will be described.

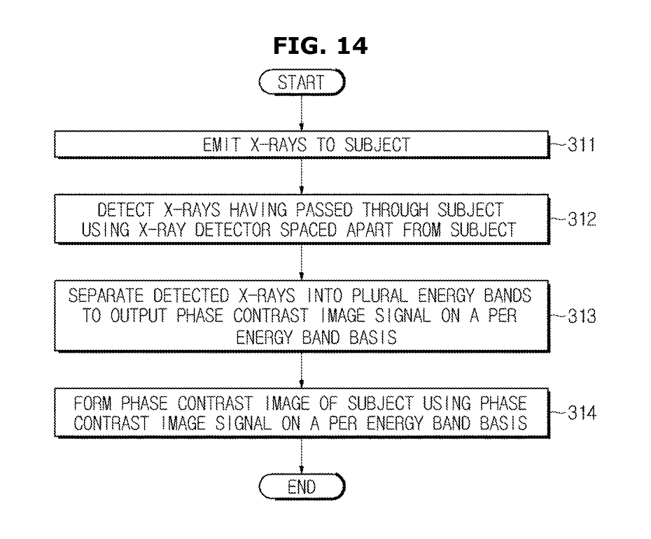

FIG. 14 is a flowchart which illustrates a control method which is executable by using an X-ray imaging apparatus according to an exemplary embodiment.

Referring to FIG. 14, first, in operation 311, X-rays are emitted toward a subject. In this case, the X-rays fall within a predetermined energy band and have high spatial coherence. Emission of X-rays which fall within a desired energy band may be accomplished by adjusting a tube voltage and a filter. Emission of X-rays having high spatial coherence may be accomplished by adjusting the size of the focal spot of an X-ray source to a range of several micrometers to dozens of micrometers (e.g., by adjusting the length of the diameter of the focal spot to within a range of between 2 micrometers and 100 micrometers), or by generating any one or more of synchrotron radiation, X-ray laser, and/or high-order harmonics.

Then, in operation 312, detection of X-rays which have propagated through the subject is implemented by using an X-ray detector which is spaced apart from the subject. A free space for propagation of X-rays which have propagated through the subject is present between the subject and the X-ray detector. As X-rays propagate through the free space, phase shift information is reflected in the intensity of X-rays. A distance between the subject and the X-ray detector may be appropriately set based on any one or more of the size of the focal spot of the X-ray source, properties of the subject, the Field of View (FOV), etc.

In operation 313, the detected X-rays are separated into a plurality of energy bands in order to output respective phase contrast image signals on a per energy band basis. To this end, photons, an energy level of which is greater than or equal to a threshold level, and which are contained in the detected X-rays, may be counted by using a photon counting detector. The range and number of the separated X-ray energy bands may be determined in consideration of the resolution or definition of a desired image and/or one or more properties of the subject.

Then, in operation 314, a phase contrast image of the subject may be generated by using the phase contrast image signals which have been acquired on a per energy band basis. Generation of the phase contrast image of the subject by using the acquired phase contrast image signals is equal to that of the above-described exemplary embodiment of the X-ray imaging apparatus 100, and thus a description of this will be omitted hereinafter. The generated phase contrast image is displayed on a display unit.

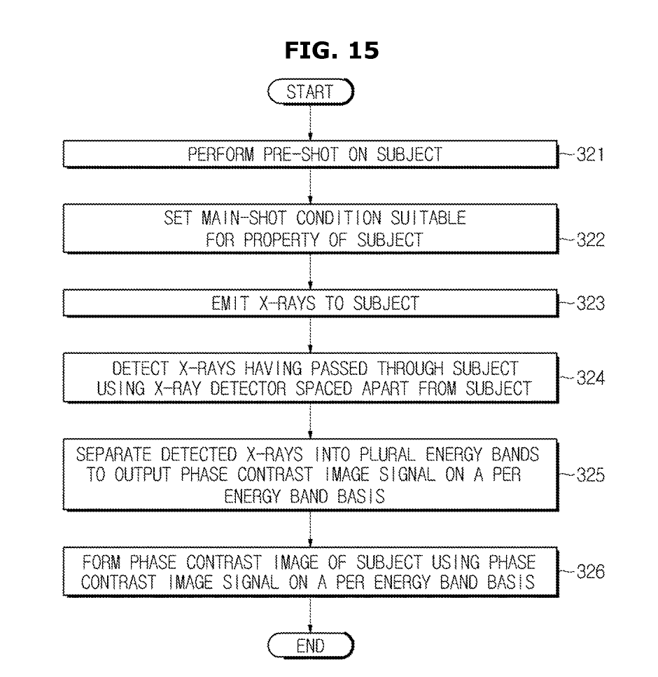

FIG. 15 is a flowchart which illustrates a control method which is executable by using an X-ray imaging apparatus that controls imaging conditions via auto-exposure control, according to an exemplary embodiment.

Referring to FIG. 15, first, in operation 321, a pre-shot is performed on a subject in order to obtain a pre-shot image of the subject. The pre-shot enables one or more X-ray imaging conditions which are suitable for properties of the subject to be set prior to a main shot, and may be performed at a low X-ray dose by adjusting tube current and X-ray exposure time.

Next, in operation 322, a pre-shot image is analyzed in order to set one or more conditions for the main shot which are suitable for properties of the subject. The set conditions may include conditions which relate to any one or more of X-ray generation and emission and distances between the X-ray source, the subject, and the X-ray detector. More specifically, at least one of X-ray generation conditions, for example, tube voltage, tube current, the target material of the anode, exposure time, and the kind of filter, may be set to be optimized to properties of the subject. In addition, as one example of X-ray emission conditions, the size of an X-ray passage region R of a collimator may be set with respect to properties of the subject in order to adjust the size of the focal spot to be suitable with respect to properties of the subject. Likewise, the conditions which relate to distances between the X-ray source, the subject, and the X-ray detector may be set to be suitable with respect to properties of the subject by analyzing a pre-shot image.

Next, in operation 323, X-rays are emitted toward the subject according to the set conditions in order to perform the main shot. In operation 324, X-rays which have propagated through the subject are detected by using the X-ray detector, which is spaced apart from the subject, and then, in operation 325, the detected X-rays are separated into a plurality of energy bands in order to output respective phase contrast image signals on a per energy band basis. Next, in operation 326, a phase contrast image of the subject may be generated by using the acquired phase contrast image signals. The generated phase contrast image may be displayed on the display unit. Alternatively, an absorptive image of the subject and the phase contrast image may be generated, or the absorptive image or the phase contrast image may be selectively generated and displayed.

As is apparent from the above description, according to an aspect of one or more exemplary embodiments, through provision of a photon counting detector that separates detected X-rays into a respective plurality of energy bands, it may be possible to acquire phase contrast image signals having different properties simultaneously without moving a detector or emitting X-rays multiple times. As a result, it may be possible to prevent deterioration in the quality of a phase contrast image due to motion artifacts, and to reduce X-ray exposure.

Although exemplary embodiments of the present inventive concept have been shown and described, it will be appreciated by those skilled in the art that changes may be made in the exemplary embodiments without departing from the principles and spirit of the present inventive concept, the scope of which is defined in the claims and their equivalents.

What is claimed is:

1. An X-ray imaging apparatus for forming a phase contrast image, the apparatus comprising:
an X-ray source which is configured to generate X-rays and to emit the generated X-rays toward a subject;
an X-ray detector which is spaced apart from the subject by at least a predetermined distance and which is configured to detect X-rays which have propagated through the subject and to separate the detected X-rays into a respective plurality of energy bands in order to acquire respective phase contrast image signals on a per energy band basis; and
an image processor which is configured to form a phase contrast image of the subject by using the acquired phase contrast image signals.

2. The apparatus according to claim 1, wherein the X-ray detector is further configured to count a number of photons which have an energy which is greater than or equal to a respective threshold energy level which corresponds to a respective one of the plurality of energy bands, from among photons included in the detected X-rays.

3. The apparatus according to claim 1, wherein the X-ray source is further configured to generate spatially coherent X-rays.

4. The apparatus according to claim 3, wherein the X-ray source has a focal spot which is used to generate the spatially coherent X-rays, wherein the focal spot has a diameter having a length of between 2 micrometers and 100 micrometers.

5. The apparatus according to claim 4, further comprising an exposure controller which is configured to control at least one condition to be applied with respect to obtaining a main shot by analyzing a pre-shot image of the subject.

6. The apparatus according to claim 5, wherein the exposure controller is further configured to determine at least one property of the subject by analyzing the pre-shot image, and to control the length of the diameter of the focal spot of the X-ray source based on the determined at least one property of the subject.

7. The apparatus according to claim 6, wherein the exposure controller is further configured to control at least one of a distance between the X-ray source and the subject and a distance between the subject and the X-ray detector based on at least one of the determined at least one property of the subject, the length of the diameter of the focal spot of the X-ray source, and a field of view of the X-ray source.

8. The apparatus according to claim 7, further comprising a fixing assembly which is vertically movably mounted between the X-ray source and the X-ray detector and which is configured to fix a position of the subject with respect to the apparatus.

9. The apparatus according to claim 8, wherein the exposure controller is further configured to control at least one of the distance between the X-ray source and the subject and the distance between the subject and the X-ray detector by moving at least one of the X-ray source, the fixing assembly, and the X-ray detector.

10. The apparatus according to claim 8, wherein the fixing assembly includes:
a support plate which is configured to support the subject; and
a compression paddle which is configured to compress the subject.

11. The apparatus according to claim 10, wherein the exposure controller is further configured to determine a thickness of the subject based on information which relates to a distance between the support plate and the compression paddle, wherein the determined at least one property of the subject include the thickness of the subject.

12. The apparatus according to claim 1, wherein the image processor is further configured to form an absorptive image of the subject by using the acquired phase contrast image signals.

13. A control method which is executable by using an X-ray imaging apparatus for forming a phase contrast image, the method comprising:
generating X-rays and emitting the generated X-rays toward a subject;
detecting X-rays which have propagated through the subject by using an X-ray detector which is spaced apart from the subject by at least a predetermined distance;
acquiring respective phase contrast image signals on a per energy band basis by separating the detected X-rays into a respective plurality of energy bands; and
forming a phase contrast image of the subject by using the acquired phase contrast image signals.

14. The method according to claim 13, wherein the acquiring the respective phase contrast image signals on a per energy band basis includes counting a respective number of photons which have an energy which is greater than or equal to a respective threshold energy level which corresponds to a respective one of the plurality of energy bands, from among photons included in the detected X-rays.

15. The method according to claim 13, wherein of the generating the X-rays includes generating spatially coherent X-rays.

16. The method according to claim 15, further comprising:
obtaining a pre-shot image of the subject; and
controlling at least one condition to be applied to a main shot by analyzing the obtained pre-shot image.

17. The method according to claim 16, wherein the controlling the at least one condition to be applied to the main shot includes determining at least one property of the subject by analyzing the pre-shot image, and controlling a length of a diameter of a focal spot of an X-ray source based on the determined at least one property of the subject.

18. The method according to claim 17, wherein the controlling the length of the diameter of the focal spot of the X-ray source includes controlling the size of the focal spot to be within a range of between 2 micrometers and 100 micrometers, in order to generate the spatially coherent X-rays.

19. The method according to claim 17, wherein the controlling the at least one condition to be applied to the main shot further includes controlling at least one of a distance between the X-ray source and the subject and a distance between the subject and the X-ray detector based on at least one of the determined at least one property of the subject, the length of the diameter of the focal spot of the X-ray source, and a field of view of the X-ray source.

20. The method according to claim 13, further comprising forming an absorptive image of the subject by using the acquired phase contrast image signals.
